(19) United States
(12) Patent Application Publication
(10) Pub. No.: US 2026/0041483 A1
(43) Pub. Date: Feb. 12, 2026

(54) FLEXIBLE MICROWAVE ABLATION DEVICE WITH DISTAL INFLATABLE BALLOON

(71) Applicant: Neuwave Medical, Inc., Madison, WI (US)

(72) Inventors: Mark THOM, Verona, WI (US); Kevin ROYALTY, Fitchburg, WI (US); Katie M. TRUTTSCHEL, Waukesha, WI (US); Matthew J. THIEL, Verona, WI (US)

(73) Assignee: Neuwave Medical, Inc., (omitted)

(21) Appl. No.: 18/799,573

(22) Filed: Aug. 9, 2024

(51) Int. Cl. A61B 18/14; A61B 18/00

(52) U.S. Cl. CPC A61B 18/1492; A61B 2018/0022; A61B 2018/00541; A61B 2018/00577

(57) ABSTRACT

A surgical system is disclosed including a probe and a balloon operatively coupled to the probe. The probe includes a shaft, an antenna extending from the shaft, and a conductor extending through the antenna and operable to deliver energy to tissue of a patient. The balloon is transitionable between an inflated state and a deflated state. In the inflated state, the balloon maintains a position of the antenna relative to the tissue and enhances energy delivery from the conductor to the tissue.

### BACKGROUND

Ablation is an important therapeutic strategy for treating certain tissues, such as benign and malignant tumors, cardiac arrhythmias, cardiac dysrhythmias and tachycardia. Some ablation systems utilize radio frequency (RF) energy as the ablating energy source. However, RF energy has several limitations, including the rapid dissipation of energy in surface tissues resulting in shallow “burns” and failure to access deeper tumor or arrhythmic tissues. Another limitation of RF ablation systems is the tendency of eschar and clot formation on the energy emitting electrodes, which limits further deposition of electrical energy.

More recently, microwave energy is being used as the ablating energy source in ablation systems. Microwave energy is an effective energy source for heating biological tissues, and is used in applications such as cancer treatment and preheating of blood prior to infusions. One advantage of microwave energy over RF is the deeper penetration into tissue, insensitivity to charring, lack of necessity for grounding, more reliable energy deposition, faster tissue heating, and the capability to produce much larger thermal lesions than RF, which greatly simplifies the actual ablation procedures.

One notable use of ablation systems is in treating lesions, such as tumors, that develop in the lungs of a patient. However, there are numerous problems associated with performing ablation procedures on the lungs. One such problem is that the lungs are non-homogenous structures including, among other things, blood vessels, air pockets, and tissue that each absorb energy from the microwave ablation devices at varying rates. This non-homogenous environment causes the thermal energy emitted from the ablation device to spread in an unpredictable manner, which may lead to the lesions not being properly treated or heathy tissue being inadvertently overheated and/or destroyed.

Accordingly, there is a continuing need for improved systems and devices for treating lesions in the lungs with ablation.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an example energy delivery system that may be used in accordance with the principles of the present disclosure.

FIG. 2 is a schematic diagram of an example probe that may be used in accordance with the principles of the present disclosure.

FIG. 3 is a schematic diagram of the probe of FIG. 2 further including a balloon in a deflated state, according to at least one aspect of the present disclosure.

FIG. 4 is the probe of FIG. 3 with the balloon in an inflated state, according to at least one aspect of the present disclosure.

FIG. 5 is the probe of FIG. 3 with the balloon in the deflated state and positioned in a bronchiole of a patient, according to at least one aspect of the present disclosure.

FIG. 6 is the probe of FIG. 5 transitioned to the inflated state, according to at least one aspect of the present disclosure.

FIG. 7 is another embodiment of the probe of FIG. 2 that includes a balloon coupled to a flexible cannula and is positioned in a bronchiole of a patient, according to at least one aspect of the present disclosure.

FIG. 8 is another embodiment of the probe of FIG. 3 with the balloon in the deflated state and further including flexible conductors in an unexpanded state, according to at least one aspect of the present disclosure.

FIG. 9 is the probe of FIG. 8 with the balloon in the inflated state and the flexible conductors in an expanded state, according to at least one aspect of the present disclosure.

FIG. 10 is another embodiment of the probe of FIG. 3 with the balloon in the inflated state and further including flexible conductors positioned on one lateral side of the balloon, according to at least one aspect of the present disclosure.

FIG. 11 is the probe of FIG. 2 further including a balloon that is coupled to a first lateral side thereof and that is in a deflated state, according to at least one aspect of the present disclosure.

FIG. 12 is the probe of FIG. 11 with the balloon in an expanded state, according to at least one aspect of the present disclosure.

FIG. 13 is another embodiment of the probe of FIG. 3 with the balloon in the deflated state, positioned in the bronchiole of FIG. 5, and further including a vent tube, according to at least one aspect of the present disclosure.

FIG. 14 is the probe of FIG. 13 with the balloon transitioned to the inflated state, according to at least one aspect of the present disclosure.

### DETAILED DESCRIPTION

The present disclosure is related to systems and methods for delivering energy to tissue for ablation operations and, more particularly, to systems and methods for controlling energy delivery to tissue through the use of inflatable balloons.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

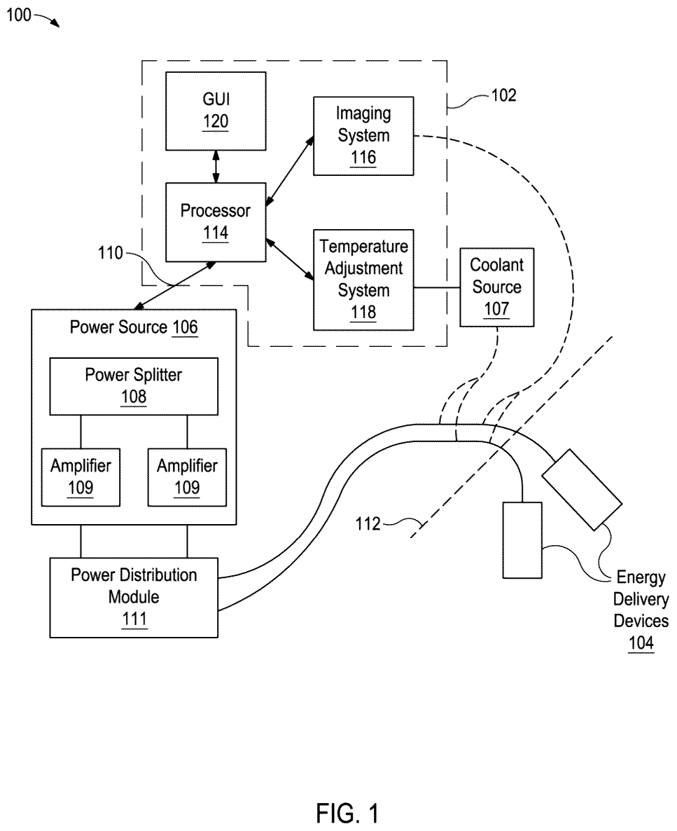

FIG. 1 is a block diagram of an example energy delivery system 100 that may incorporate the principles of the present disclosure. As illustrated, the energy delivery system 100 (hereafter “the system 100”) includes a control system 102 and one or more energy delivery devices or “ablation probes” 104 (two shown) designed to deliver (emit) energy to a target tissue region of a patient. An example ablation probe 104 that can be used with the energy delivery system 100 is described in U.S. Patent Application Publication No. 2021/0282851, titled “ENERGY DELIVERY SYSTEMS AND USES THEREOF”, which published on Sep. 16, 2021, the disclosure of which is hereby incorporated by reference in its entirety herein.

The system 100 further includes a power source or generator 106 communicably coupled to the control system 102 and the ablation probes 104 to direct, control, and deliver (provide) electrical power thereto. In some applications, the power source 106 may include a power splitter 108 that receives power from an external power source (e.g. a wall outlet) and directs power to one or more amplifiers 109, which amplify the voltage, current, or power from the power splitter 108. While two amplifiers 109 are shown, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which provides strain relief to the cabling extending from the amplifiers 109 to the ablation probes 104. The power distribution module 111 may be coupled to a structure in the operating room, such as a surgical bed, and may house connection hardware of the probes 104.

The components of the system 100 are connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The control system 102 is configured to monitor, control, and provide feedback concerning operation of the system 100. As illustrated, the control system 102 includes at least a processor 114, an imaging system 116, and a temperature adjustment system 118. The control system 102 may further include a graphical user interface (GUI) 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region to allow a user (operator) to operate and/or monitor the components of the system 100.

The processor 114 may be provided within a computer system or module, which may include software or software instructions executable by the processor 114 to carry out functions and operations of the system 100. The software may be stored on a computer memory or computer memory device comprising any storage media readable by the processor 114. Examples of the computer memory include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 114. Examples of computer readable media include, but are not limited to, optical discs, magnetic disks, magnetic tape, solid-state media, and servers for streaming media over networks.

Based on instructions provided by the software, the processor 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The processor 114 interacts with the ablation probes 104 to raise or lower (e.g., tune) the amount of energy delivered to the tissue region. The processor 114 may also be configured to prime coolants for distribution into the ablation probes 104 such that the coolant is delivered at a desired temperature. In some applications, the type of tissue being treated is inputted into the software for purposes of allowing the processor 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected ablation sizes (as described in more detail herein) may be based on tissue type. In such embodiments, the processor 114 may not control power delivery based on tissue type. In yet other embodiments, the processor 114 generates a chart or diagram based upon a particular type of tissue or tissue region displaying characteristics useful to a user of the system. The processor 114 may allow a user to choose power, duration of treatment, different treatment algorithms for different tissue types, simultaneous application of power to multiple probes 104, coherent and incoherent phasing, etc. The processor 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

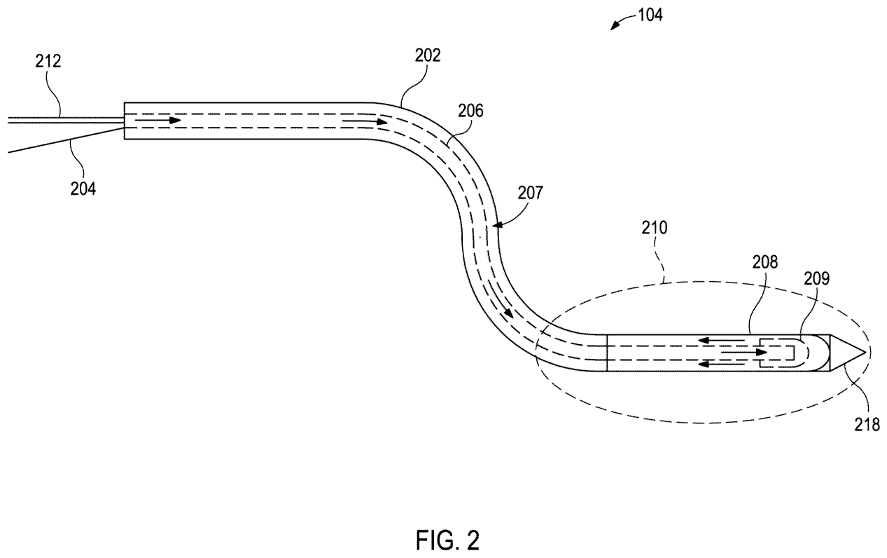

FIG. 2 is a schematic diagram of an example probe 104 that may incorporate the principles of the present disclosure. As indicated above, the ablation probe 104 may be configured to deliver (emit) energy (e.g., microwave energy, radiofrequency energy, radiation energy) to a target tissue region. As illustrated, the ablation probe 104 includes a flexible, elongate shaft or probe cannula 202 that enables the probe 104 to navigate a working channel of a bronchoscope, for example. In some embodiments, the flexible probe cannula 202 is made of a helically wound ribbon that enables the probe cannula 202 to be flexible. In some embodiments, the helically wound ribbon is a silver-plated spiraled copper ribbon or a functional equivalent thereof. In some embodiments, the probe cannula 202 includes an outer layer that serves to provide structural stability to the probe cannula 202. The outer later may comprise, for example, a polyester jacket and/or a polyethylene terephthalate (PET) heat shrink layer.

A cable or cable assembly 204 may be configured to convey electrical power to the probe 104. The cable assembly 204 may extend from the power source 106 (FIG. 1), for example, and may provide the power sufficient to operate the ablation probe 104. An antenna 208 is provided at the distal end of the probe cannula 202 and sized to receive a conductor 206 that extends through the flexible probe cannula 202 to the antenna 208. Electrical power from the cable assembly 204 is provided to the conductor 206 and emitted from the conductor 206 at the antenna 208 to a target tissue region to thereby generate an ablation zone 210.

The material of the antenna 208 is a durable dielectric. In some applications, the antenna 208 is made of a polymer, such as methyl fluoroacetate (MFA), or a functional equivalent of MFA. In some applications, the conductor 206 is made of copper or a functional equivalent of copper. In some embodiments, the conductor 206 comprises a first portion that extends through the cannula 202 and a second portion extending from the first portion through the antenna 208 and that is comprised of a second material different than the first material. In such embodiments, the first material may be more flexible than the second material to enable the first portion, which extends through the flexible cannula 202, to bend more than the second portion which extends through the antenna 208. In some embodiments, the antenna 208 includes a cap 209 coupled to a distal end of the conductor 206 and that serves as a load point for the received electrical power from the cable assembly 204. The cap 209 may be made of brass or a functional equivalent of brass.

In some applications, a cooling tube 212 is configured to convey a cooling fluid to the probe 104 from the coolant supply 107 (FIG. 1). The control system 102 may be configured to control conveyance of the cooling fluid into and out of the probe cannula 202 to help regulate a temperature of the antenna 208 and the ablation zone 210. Example cooling fluids include, but are not limited to, water, glycol, air, inert gasses, carbon dioxide, nitrogen, helium, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

In some embodiments, the conductor 206 is hollow and the cooling tube 212 is fluidically coupled to the conductor 206 such that the cooling fluid is conveyable from the coolant source 107 (FIG. 1) to the antenna 208 via the conductor 206. In some embodiments, the probe 104 further defines a return path 207 between the probe cannula 202 and the conductor 206 to convey the cooling fluid provided to the antenna 208 out of the probe 104. The flow path of the cooling fluid through the conductor 206 and the return path 207 out of the probe 104 are shown in FIG. 2 with arrows extending within the cannula 202. In some embodiments, the cap 209 serves to redirect the cooling fluid flow from the conductor 206 toward the return path 207. In some embodiments, a monofilament (not shown) is helically wrapped around the conductor 206 to maintain a gap between the conductor 206 and the probe cannula 202 (i.e., the return path 207).

The probe 104 may further include a core tube (not shown) concentrically disposed between the conductor 106 and the probe cannula 202 and which functions to seal the return path 207. In some embodiments, the core tube may comprise helically-wrapped monofilament and may be made of a polymer, such as methyl fluoroacetate (MFA), or a functional equivalent of MFA. The core tube may further contribute to the spacing within the probe 104 and the dielectric properties of the conductor 104.

In some applications, the ablation probe 104 may further include a stylet tip or “stylet” 218 positioned at the distal end of the antenna 208 and otherwise forming the distal end of the ablation probe 104. In some embodiments, the stylet 218 includes a sharp tip that is designed to penetrate tissue. In other embodiments, the stylet 218 includes a blunt tip to navigate working channels within a patient. The stylet 218 may be made of a variety of rigid or hardened materials including, but not limited to, a hardened resin, a metal (e.g., titanium or an equivalent of titanium, stainless steel, etc.). In at least one application, the stylet 218 may be brazed to zirconia or an equivalent of zirconia. In such applications, the stylet 218 may comprise an extension of a metal portion of the antenna 208 and may be electrically active.

Referring again to FIG. 1, the control system 102 further includes the imaging system 116, which is in communication with the processor 114 comprises one or more imaging devices. Example imaging devices include, but are not limited to, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

In some applications, the system 100 provides software configured for use with imaging equipment of the imaging system 116, such as CT, MRI, and ultrasound, and generate two-dimensional (2D) and or three-dimensional (3D) images viewable by a user on the GUI 120. In some embodiments, the imaging equipment software allows a user to make predictions based upon known thermodynamic and electrical properties of tissue, vasculature, and location of the antenna(s) 208 (FIG. 2). In some embodiments, the imaging software allows the generation of a 2D or 3D map of the location of a tissue region (e.g., tumor, arrhythmia), location of the antenna(s) 208, and to generate a predicted map of the ablation zone 210 (FIG. 2).

In some applications, the imaging system 116 may be configured to monitor ablation procedures, such as monitoring the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The power source 106 may be configured to supply the energy required to operate the system 100. The power source 106 is also configured to supply energy to the ablation probes 104, such as microwave energy, radiofrequency energy, radiation, cryo energy, electroporation, high intensity focused ultrasound, or any combination thereof. In accordance with principles of the present disclosure, the power source 106 supplies microwave energy to the ablation probes 104 for purposes of tissue ablation. More specifically, power may be supplied to the ablation probes 102, but the microwave energy is generated in a microwave generator and sent to the antenna 208. In some applications, the power source 106 may include one or more energy generators configured to provide as much as 140-150 watts of microwave power of a frequency of from 915 MHz to 5.8 GHz, although the present invention is not so limited. The power splitter 108 may comprise a power distribution system operable to distribute the energy from the power source 106 to the ablation probes 104. The power splitter 108 may be configured to provide varying energy levels to different regions of the ablation probes 104.

The temperature adjustment system 118 may be configured to use coolant systems and cooling fluids to help reduce undesired heating within and along the ablation probes 104. In particular, the temperature adjustment system 118 may include the coolant supply 107 (FIG. 1) and the cooling tube 212 (FIG. 2) and may communicate with each probe 104 to control conveyance of the cooling fluid into and out of the probe cannula 204 (FIG. 2), and thereby help regulate a temperature of the antenna 208 (FIG. 2) and the ablation zone 210 (FIG. 2).

The temperature adjustment system 118 may also be configured to continuously or intermittently monitor the real-time temperature of the ablation probes 104. In such embodiments, the temperature adjustment system 118 may communicate with one or more temperature sensors (e.g., thermocouples) terminating at various points along the probe cannula 204 (FIG. 2) and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210 (FIG. 2), such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length. The control system 102 can receive the temperature measurements from the temperature sensors and control the cooling fluid flow to the probe 104 through the conductor 206 based on the measurements.

The temperature adjustment system 118 may also be configured to monitor the temperature of a tissue region (e.g., tissue being treated, surrounding tissue). This may prove advantageous in helping to determine the status of the procedure (e.g., the end of the procedure). The temperature adjustment system 118 may communicate with the processor 114 to provide real-time temperature information to a user and display such measurements on the GUI 120. In at least one embodiment, based on the temperature data obtained by the temperature adjustment system 118, the processor 114 may be configured to autonomously adjust operation of the system 100 appropriately.

As described herein, there are numerous obstacles associated with performing ablation procedures on the lungs. For example, the lungs are non-homogenous structures, which can result in the generated ablation zone 210 spreading in an unpredictable manner, which may lead to the lesions not being properly treated and/or heathy tissue being inadvertently overheated and destroyed.

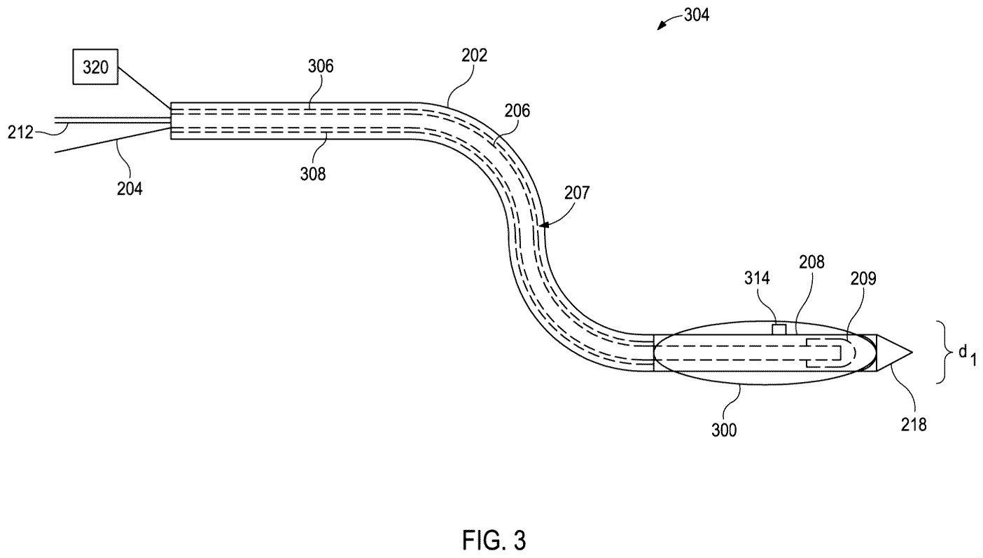

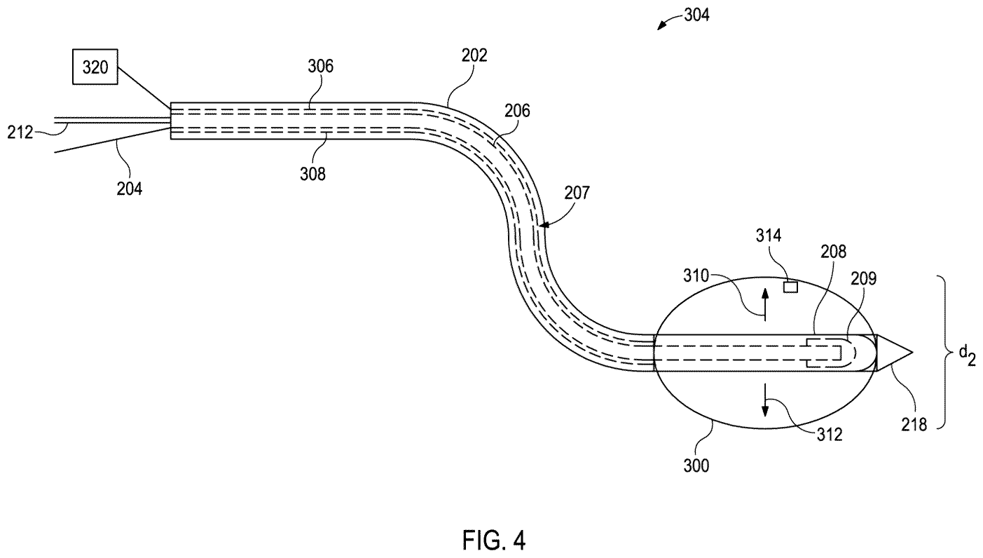

FIGS. 3 and 4 are schematic diagrams of an example probe 304, according to at least one aspect of the present disclosure. The probe 304 is substantially similar to the probe 204 (FIG. 2) with like numbers used in the figures to indicate their similarities. The probe 304, unlike probe 204, further includes an inflatable balloon 300. In some embodiments, the balloon 300 is made of a thermoplastic, such as polypropylene, polyethylene, polyvinyl chloride, polystyrene, polyethylene terephthalate, or polycarbonate, or combinations thereof. In the illustrated embodiment, the balloon 300 is operatively coupled to the antenna 208. In other embodiments, however, the balloon 300 may be operatively coupled to the probe 304 at a location proximal to the antenna 208, such as on the flexible cannula 202.

The balloon 300 is fluidically coupled to a fluid source 320 and receives a fluid therefrom to transition the balloon 300 from a deflated state, as shown in FIG. 3, in which the balloon 300 defines or exhibits a first diameter d₁ around the antenna 208, to an inflated state, as shown in FIG. 4, in which the balloon defines or exhibits a second diameter d₂ around the antenna 208 and greater than the first diameter d₁. In some embodiments, the fluid source 302 is the same fluid source that provides coolant to the cooling tube 212 (i.e., the coolant supply 107). In other embodiments, however, the fluid source 302 may alternatively be a fluid source separate from the coolant source. In some embodiments, the fluid source 302 includes a syringe graspable by a clinician (operator) and actuatable to inject a fluid into the balloon 300. In some embodiments, the fluid may be a gas, such as air or carbon dioxide, for example. In other embodiments, the fluid may be a liquid, such as water or saline, for example. In such embodiments, the fluid may be referred to as a “matching” fluid, which refers to a fluid preferably having a dielectric constant (e.g. 30-80) similar or greater than that of the target tissue. The matching fluid preferably has a stable dielectric constant over an expected range of temperatures or is controlled to a tight range of temperatures via the temperature control system 118 or via constant flow. The matching fluid may provide a consistent contact and dielectric load to the antenna 208, allowing the antenna 208 to be optimized for a known load. The matching fluid may absorb and distribute some of the generated energy, creating a more even temperature distribution. Cooling effects of the fluid may help prevent charring and desiccation of the target tissue (temperatures greater than 100° C., for example), which may help keep the target tissue dielectric properties in a range tuned to the antenna 208, and also may have benefits for the patient for post ablation healing.

The balloon 300 is fluidically coupled to the fluid source 302 via a fluid input line 306 that extends through the flexible probe cannula 202, alongside the conductor 206. In embodiments where the conductor 206 is a hollow cannula, the fluid input line 306 may extend within the interior of the conductor 206. In other embodiments, the fluid input line 306 may extend outside of the cannula 202 and under a layer of heat shrink material. In other embodiments, the fluid input line 306 may extend parallel to and along the outside (exterior) of the conductor 206. In some embodiments, the fluid may be delivered to and evacuated from the balloon 300 via the fluid input line 306. In other embodiments, however, the probe 104 may further include a fluid output line 308 that runs alongside the conductor 206 (e.g., either within or along the exterior). In such embodiments, the fluid output line 308 may be fluidically coupled to the balloon 300 to convey the fluid provided to the balloon 300 back to the fluid source 302 or a fluid sink. The fluid input and outline lines 306, 308 enable a user to provide a circulating flow of fluid to the balloon 300, as will be described in more detail below. In some embodiments, the fluid input and output lines 306, 308 extend along the outside of the flexible cannula 202.

In some embodiments, the control system 102 is in operable communication with the fluid source 302 to control an amount and/or flow rate of fluid to the balloon 300 from the fluid source 302. In some embodiments, a user can provide an input to the control system 102, such as at the GUI 120, for example. Based on the user input, the control system 102 causes the fluid source 302 to provide the fluid to the balloon 300. In some embodiments, a user manually controls the amount and/or flow rate of fluid to the balloon 300 from the fluid source 302 by interacting and/or manually actuating the fluid source 302, such as when the fluid source 302 is a syringe or another manual injection means.

In some embodiments, the amount and/or flow rate of fluid that is to be provided to the balloon 300 is stored in a memory and retrievable by the processor 114. The amount and/or flow rate of fluid stored in the memory may be predefined, and may be based on the tissue to be ablated or the location of the balloon 300. In some embodiments, the amount and/or flow rate of fluid to provide to the balloon 300 is based on a user input to the control system 102, such as at the GUI 120.

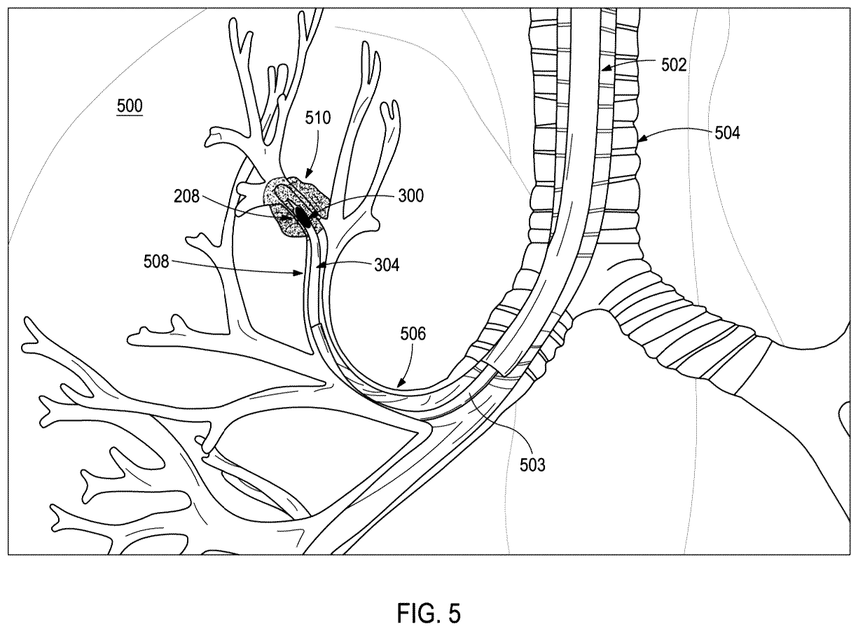

In some embodiments, the balloon 300 can be circumferentially situated (arranged) around the antenna 208 such that the balloon 300 expands radially outward from the probe 304 in all radial directions. In the illustrated embodiment, the balloon 300 is shown expanding in at least a first radial direction 310 (FIG. 4) and a second radial direction 312 (FIG. 4) opposite the first radial direction 310. In such embodiments, the balloon 300 can function to center the antenna 208, such as within a bronchiole 508 (FIG. 5), to create a more uniform ablation zone 210 therewithin.

In at least one embodiment, the balloon 300 may include a force sensor 314 that communicates with the control system 102 (either wired or wirelessly) and is operable to sense an amount of force that the balloon 300 exerts on surrounding tissue. Based on the measured force from the force sensor 314, the control system 102 can adjust the amount and/or flow rate of fluid provided to the balloon 300. For example, when the detected force approaches, reaches, or exceeds an upper force threshold, indicating that the balloon 300 is applying too much force to the surrounding tissue, the control system 102 can control (decrease) the amount of fluid in the balloon 300, or flow rate of fluid to the balloon 300, based on the detection. As another example, when the detected force approaches, reaches, or drops below a lower force threshold, indicating that the balloon 300 may not have been expanded enough, the control system 102 can control (increase) the amount of fluid in the balloon 300, or flow rate of fluid to the balloon 300, based on the detection.

The inclusion of the balloon 300 to the probe 304 provides numerous advantages with respect to ablating tissue, such as lung tissue. As one example, as discussed above, one problem associated with performing ablation procedures on the lungs is that the lungs are non-homogenous in nature, causing the generated ablation zone 210 to spread in an unpredictable manner. The balloon 300 may prove advantageous in helping to control the ablation zone 210 created by the antenna 208.

Referring now to FIG. 5, the probe 304 can be positioned within a lung 500 of a patient. More specifically, a bronchoscope 502 can be positioned (steered) through the patient's trachea 504, a bronchi 506, and into a bronchiole 508 that includes a lesion (tumor) 510. With the balloon 300 in the deflated state (FIG. 3), the probe 304 can be pushed (advanced) through an access sheath 503 of the bronchoscope until the antenna 208 is positioned adjacent to the lesion 510. The access sheath 503 may be a part of the bronchoscope 502 (i.e., the bronchoscope 502 may comprise or form part of the access sheath 503), or may be a separate component from the bronchoscope 502 and the probe 304. In some embodiments, positioning the antenna 208 relative to the lesion 510 can be determined using the imaging system 116.

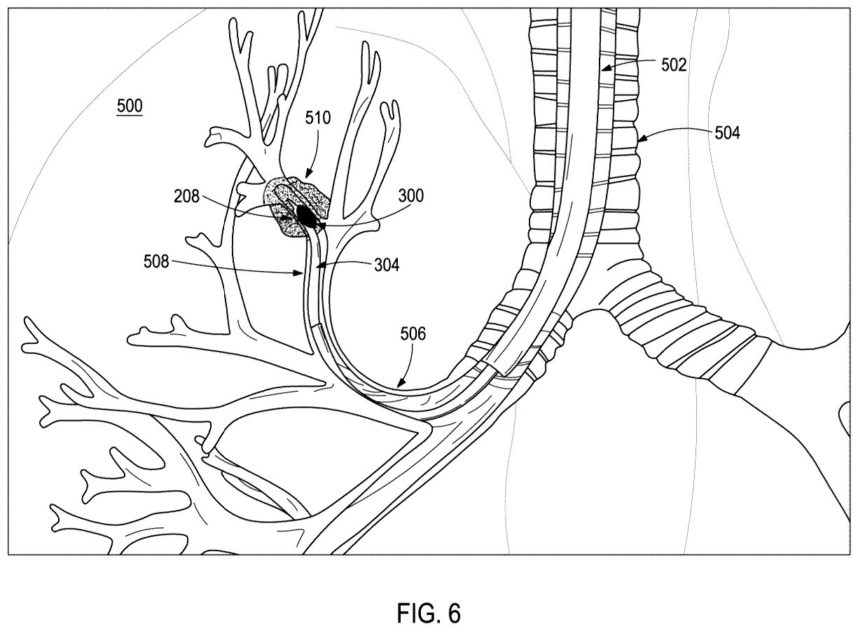

Referring now to FIG. 6, with the antenna 208 in a desired position (location), such as adjacent the lesion 510, the balloon 300 can then be inflated with the fluid from the fluid source 320 (FIGS. 3-4) to transition the balloon 300 from the deflated state (FIG. 3) to the inflated state (FIG. 4). The balloon 300 is shown in FIG. 6 in the inflated state. In the inflated state, the balloon 300 expands and frictionally engages the surrounding bronchiole 508, thereby maintaining a position of the antenna 208 and preventing unwanted movement thereof during the ablation procedure. In some embodiments, in the inflated state, the diameter d₂ (FIG. 4) of the balloon 300 is greater than or equal to the diameter of the bronchiole 508 to fully occlude the bronchiole 508. In other embodiments, however, in the inflated state, the diameter d₂ of the balloon 300 is greater than or equal to the diameter of the bronchi 506 to fully occlude the bronchi 506. In at least one embodiment, the diameter d₂ of the balloon 300 is at least 10 mm when in the inflated state.

As shown in FIGS. 3-6, the balloon 300 can be positioned at the distal end of the probe 304, and more specifically, directly over the antenna 208 of the probe 304. In the inflated state, the fluid filled balloon 300 surrounds the antenna 208 to provide the antenna 208 with a more homogeneous, predictable, and consistent media to emit its energy as compared to the non-homogenous environment of the lung 500. This homogeneous, predictable, and consistent media causes the generated ablation zone 210 to be more consistent and predictable in manner, giving the user more control over what is being ablated when operating the probe 304. Moreover, this homogeneous, predictable and consistent media also makes the antenna 208 less sensitive to the anatomical position in the lung 500, increases the conduction of energy into the lesion 510, and increases the efficiency of the energy transfer from the antenna 208.

As the antenna 208 operates and generates the ablation zone 210 (FIG. 2), fluid can be circulated to the balloon 300 via the fluid input and output lines 306, 308 (FIGS. 3-4) to maintain a near consistent (constant) temperature of fluid in the balloon 300. This may prove advantageous in helping to prevent the fluid in the balloon 300 from overheating or boiling, and further increases the predictability and consistency of energy transfer from the antenna 208, while also preventing the heated tissue from charring or desiccating. This also serves a two-fold benefit of preserving the dielectric properties of the tissue, leading to better antenna 208 performance, while also potentially reducing the formation of long lasting scar tissue.

Furthermore, when positioned within the bronchiole 508, for example, inflating the balloon 300 to the inflated state, as shown in FIGS. 4 and 6, may block airflow to the bronchiole 508. Blocking the airflow may cause the lung 500 to collapse around the balloon 300 and the antenna 208, thereby increasing the amount of surface area of the probe 304 that is in contact with the bronchiole 508. This increased surface contact (as opposed to the probe 304 being suspended in the air within the bronchiole 508) increases the amount of energy deposition from the antenna 208 into the lesion 510 when the ablation zone 210 is generated.

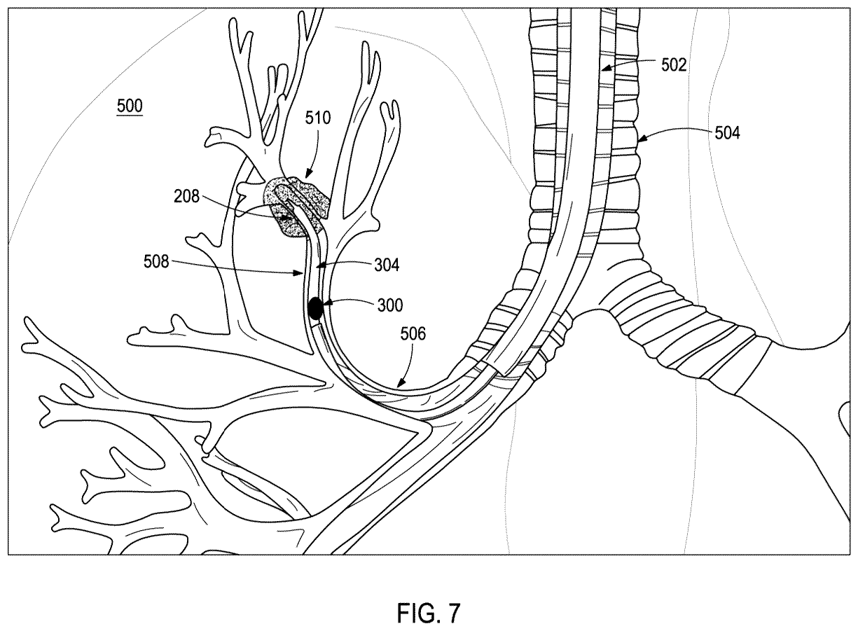

Referring now to FIG. 7, in some embodiments the balloon 300 can be positioned proximal to the antenna 208, such as operatively coupled to the flexible cannula 202. In such embodiments, the balloon 300 may be attached to the distal end of the flexible cannula 202, for example. In yet other embodiments, the balloon 300 can be positioned proximal to the probe 304 itself, such as on the distal end of the access sheath 503.

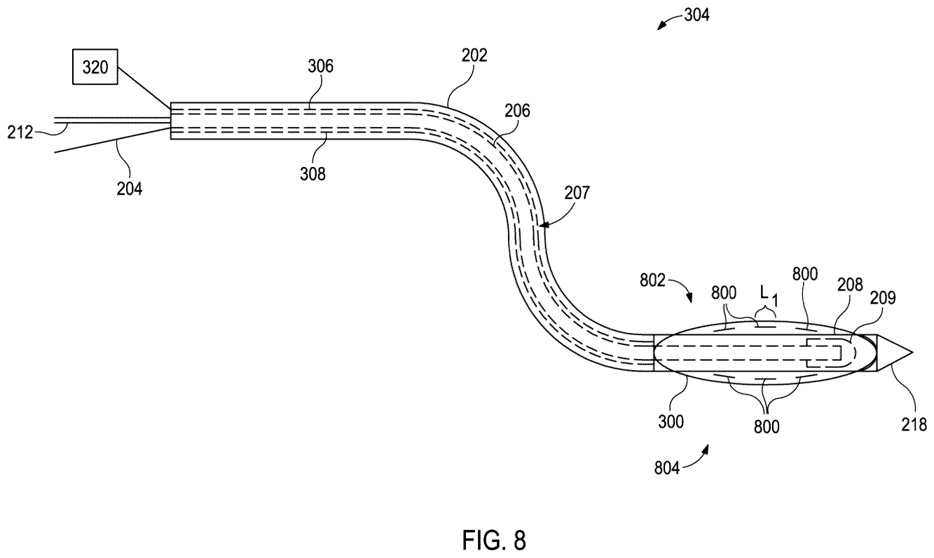

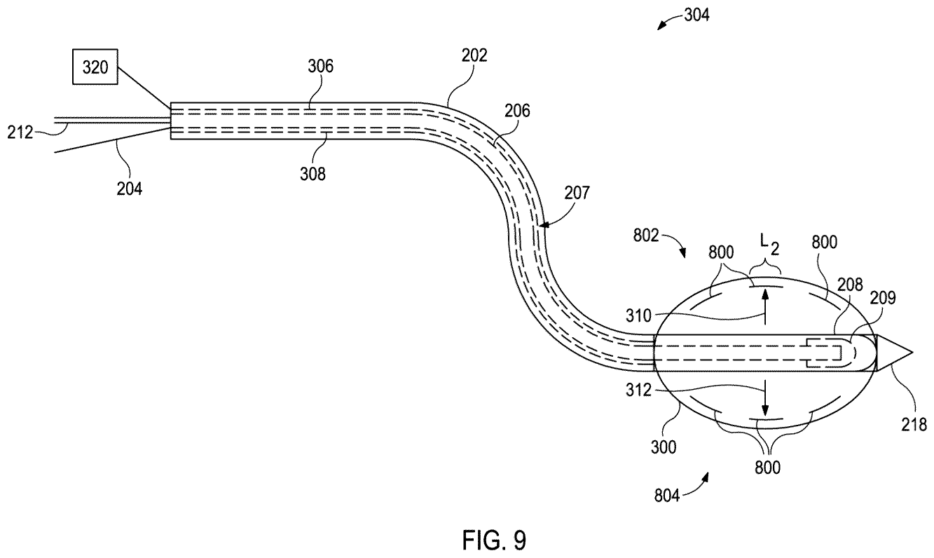

FIGS. 8 and 9 depict an alternative embodiment of the balloon 300, according to one or more embodiments of the present disclosure. More specifically, in some embodiments, the balloon 300 may include metallic elements or filaments that may help control the energy emitted from the conductor 206 at the antenna 208. For instance, in some embodiments, the balloon 300 may include one or more flexible metallic elements 800 that are expandable as the balloon 300 expands between the deflated and inflated states. The metallic elements 800 may be printed onto the surface of the balloon 300 (e.g., printed conductive inks), or may be deposited on the balloon via chemical vapor deposition (CVD) or physical vapor deposition (PCD). In some embodiments, the metallic elements 800 may be helically wound around the balloon 300.

The metallic elements 800 may be expandable between an unstretched state, as shown in FIG. 8, and corresponding to the deflated state of the balloon 300, and a stretched state, as shown in FIG. 9, and corresponding to the inflated state of the balloon 300. In the unstretched state, the metallic elements 800 may each extend a first length L₁ along the balloon 300. In the stretched state, the metallic elements 800 may each extend a second length L₂ along the balloon 300, which is greater than the first length L₁.

In some embodiments, the metallic elements 800 are made of a ferromagnetic material and in electrical communication with the power source 106 (FIG. 1) to receive energy therefrom. In such embodiments, the metallic elements 800 may communicate with the power source 106 via a dedicated wire (e.g., a coaxial cable) or wires that extend from the power source 106 to the metallic elements 800 via the flexible cannula 202. In other embodiments, however, the metallic elements 800 may communicate with the power source 106 via the conductor 206. In such embodiments, the metallic elements 800 are coupled to the conductor 206 with a wire or wires that extend from the conductor 206 to the metallic elements 800. In other embodiments, the metallic elements 800 are coupled to the conductor 206 via a resonant structure therebetween.

In some embodiments, the metallic elements 800 are operable to emit supplemental energy to tissue surrounding the antenna 208, such as the lesion 510 (FIG. 5). In some embodiments, the metallic elements 800 control or direct the energy emitted from the conductor 206 to the surrounding tissue. More specifically, in at least one embodiment, the metallic elements 800 may be configured to draw energy emitted from the antenna 208 in the direction of the metallic elements 800. In other embodiments, the metallic elements 800 are selectively arranged on the balloon 800 to block microwave energy emitted from the antenna 208 in the direction of the metallic elements 800, similar to how a Faraday cage operates. In some embodiments, a first portion of the metallic elements 800 may be arranged on a first side 802 of the balloon 300 and a second portion of the metallic elements 800 may be arranged on a second side 804 of the balloon 300. In some such embodiments, the metallic elements 800 arranged on the first side 802 may serve to direct (draw) energy toward the first side 802, while the metallic elements 800 arranged on the second side 804 may serve to block energy flow toward the second side 804. Accordingly, the metallic elements 800 may co-operatively function to direct energy toward only one side of the balloon 300—the first side 802—which gives the user more control over energy emission from the antenna 208.

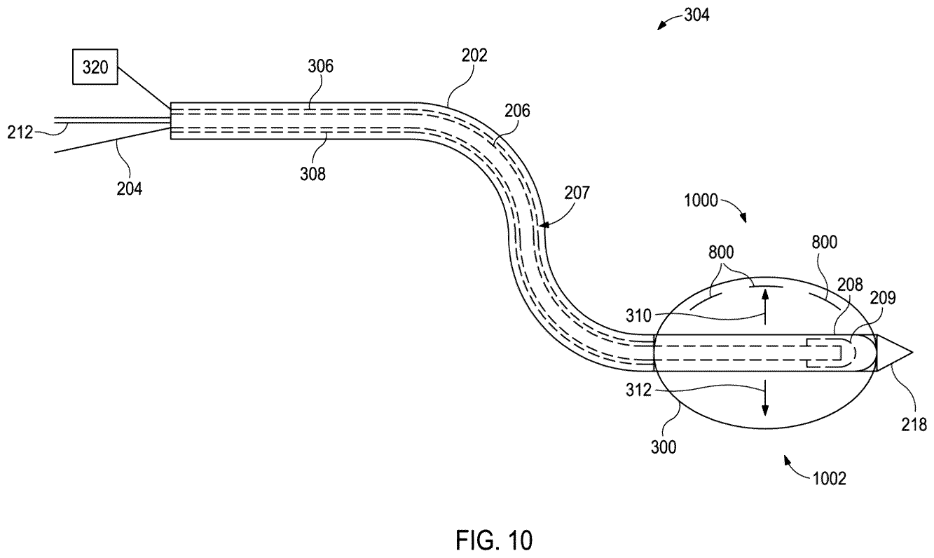

FIG. 10 depicts an alternative embodiment of the balloon 300, according to one or more embodiments of the present disclosure. More specifically, in some embodiments, the balloon 300 may include a first side 1000 that includes one or more metallic elements 800, and a second side 1002 opposite the first side 1000 that lacks metallic elements 800. The metallic elements 800 may be arranged on the first side 1000 such that energy emitted from the conductor 206 at the antenna 208 is directed toward the first side 1000 as opposed to the second side 1002. Accordingly, the arrangement of the metallic elements 800 along the balloon 300 enables a user to be more selective of where energy is radially emitted from the antenna 208, which is particularly useful when a lesion is only one side of a bronchiole, for example.

Alternatively, the metallic elements 800 may be operable to block energy emitted from the conductor 206 at the antenna 208 (i.e., a Faraday cage-like arrangement). For instance, the metallic elements 800 on the first side 1000 may block energy emitted from the antenna 208 such that the energy emitted from the antenna 208 is only directed toward the second side 1002. Accordingly, the arrangement of the metallic elements 800 along the balloon 300 can enable a user to block (protect) portions of healthy tissue from energy emitted from the antenna 208.

The metallic elements 800 may be embedded in the balloon 300 and serve as a choke proximal to the radiating portion of the antenna 208 to prevent proximal ablation growth. The metallic elements 800 may form a ¼ wave choke in the balloon 300 shorting to the cannula 202 at a proximal end of the balloon 300. The conductive section of balloon 300 may extend distally on the surface of the balloon 300 a length corresponding to a ¼ wavelength of the microwave signal in the fluid of the balloon 300. By placing the metallic elements 800 (choke) on the surface of the balloon 300, the choke can achieve a larger diameter than monolithic devices in which the choke is built into a probe wall. Other choke structures such as a floating choke are also envisioned. A function of the metallic elements 800 may be to prevent proximal ablation growth that occurs as the field pattern of the antenna 208 elongates due to changes in the tissue dielectric properties or due to an unbalanced antenna 208 design.

In some embodiments, controlling energy emission from the antenna 208 using the metallic elements 800 may be dictated by and otherwise based on the length of the individual metallic elements 800. More specifically, in the unstretched state (FIG. 8) where the metallic elements 800 have a first length L₁, the metallic elements 800 cover a first longitudinal area (span) of the balloon 300, and thus control energy emission from the antenna 208 by a first amount or quantity. In the expanded state (FIG. 9) where the metallic elements 800 have a second length L₂ greater than the first length L₁, the metallic elements 800 cover a second longitudinal area of the balloon 300 greater than the first longitudinal area, and thus control energy emission from the antenna 208 a second amount greater than the first amount. Accordingly, a user and/or the control system 102 can adjust the energy regulation provided by the metallic elements 800 by varying the balloon 300 between the deflated state (FIG. 8) and the inflated state (FIG. 9).

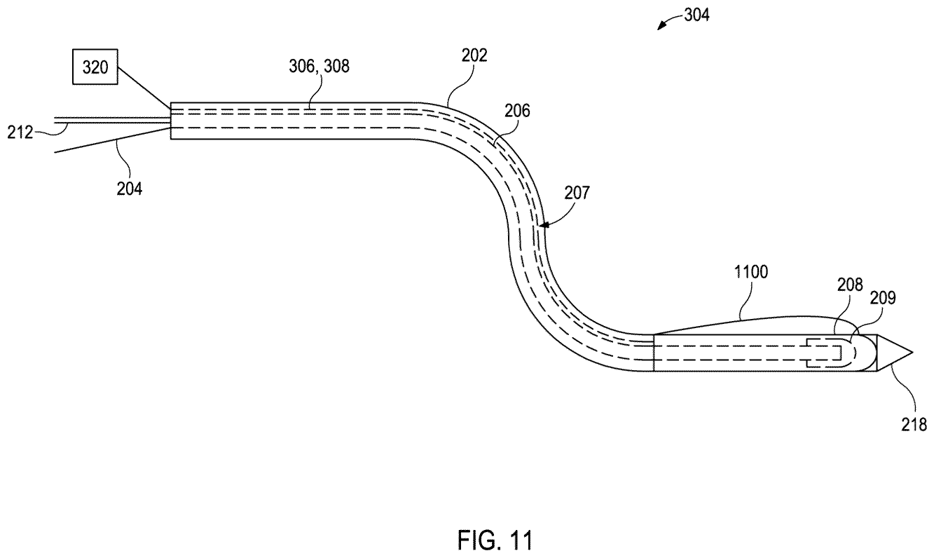

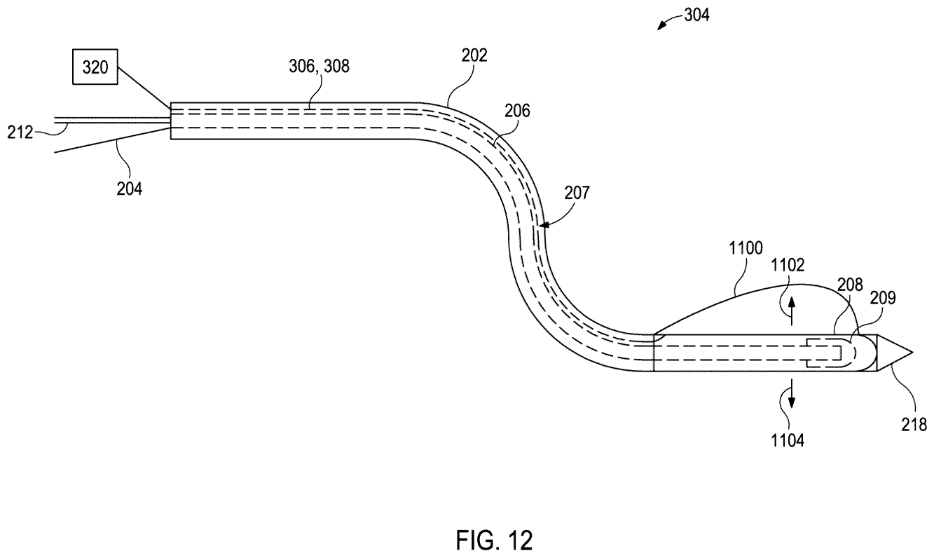

FIGS. 11 and 12 depict an alternative embodiment of the probe 304, according to one or more additional embodiments of the present disclosure. In some embodiments, as illustrated, the probe 304 can include a balloon 1100 that expands from only one side of the probe 304. More specifically, the balloon 1100 may be transitionable from a deflated state, as shown in FIG. 11, to an expanded state, as shown in FIG. 12, in which the balloon 1100 expands from only one side of the probe 304 in a first radial direction 1102 (FIG. 12). By expanding from only one side of the probe 304, the balloon 1100 can engage an anatomical structure, such as a wall of the bronchiole 508 (FIG. 5), thereby forcing the antenna 208 to move in a second radial direction 1104 opposite the first radial direction 1102 and into direct contact with an opposing wall of the anatomical wall. Bringing the antenna 208 into direct contact with the opposing wall of the anatomical structure, as opposed to being suspended within an airway thereof, can increase the amount of thermal energy that is conducted into the anatomic structure when the ablation zone 210 is generated. Furthermore, in the inflated state, the balloon 1100 can frictionally engage the anatomical structure to maintain a position of the antenna 208 and prevent unwanted movement thereof during the ablation procedure.

The balloon 1100 may also provide the user with more directional control over energy emitted from the antenna 208. For example, a lesion may be located on one side of the bronchiole 508 (FIG. 5) and healthy tissue may be located on an opposite wall of the bronchiole 508. The user can manipulate (e.g., rotational position) the probe 304 such that the balloon 1100 confronts (faces) the healthy tissue wall. The balloon 1100 can then be expanded to the inflated state (FIG. 12) in the first radial direction 1102 to force the antenna 208 toward the wall of the bronchiole 508 that includes the lesion and, thus, away from the healthy tissue. This enables the user to direct the energy emitted from the antenna 208 more toward the lesion than the healthy tissue. In addition, the balloon 1100 can include metallic elements 800 (FIG. 8) to further control energy emission from the antenna 204 toward the lesion.

In some instances, referring again to FIGS. 5 and 6, inflating the balloon 300 positioned within the bronchiole 508 to the inflated state may block airflow to the bronchiole 508, which may cause the lung 500 to collapse around the balloon 300 and the antenna 208. In some instances, it may be desirable to prevent the lung 500 from collapsing around the balloon 300 when inflated to the inflated state. For example, navigation and imaging modalities that rely on registration to a pre-procedure CT scan may not function properly when the lung 500 is collapsed.

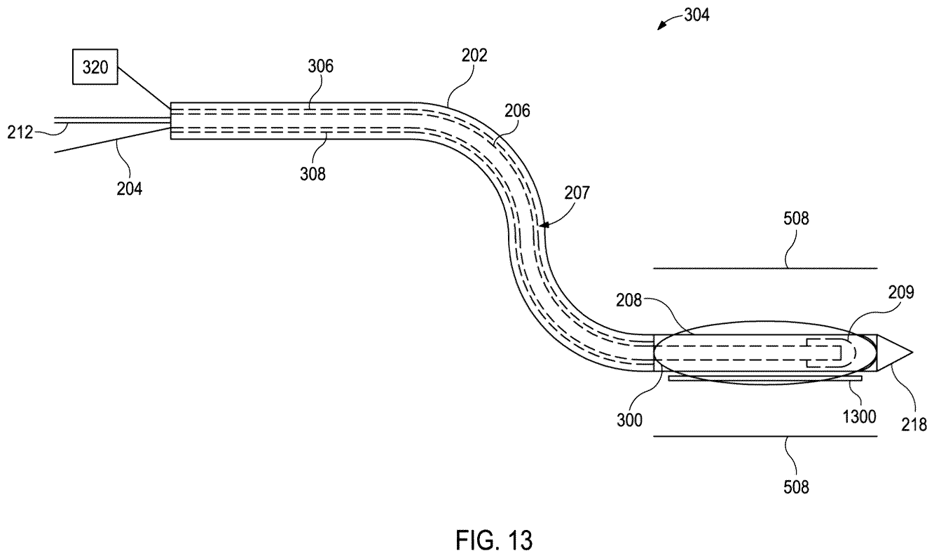

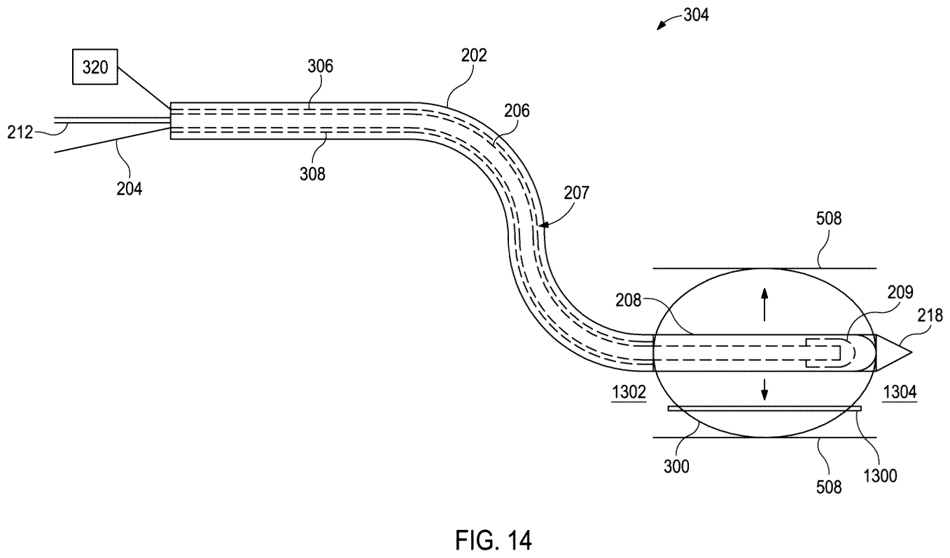

Accordingly, FIGS. 13 and 14 depict an alternative embodiment of the balloon 300, according to one or more embodiments of the present disclosure. More specifically, the balloon 300 may include a vent tube 1300 extending through the balloon 300 that allows airflow therethrough. The balloon 300 may be positioned within the bronchiole 508 and may receive a fluid from the fluid source 320 to transition the balloon 300 from a deflated state, as shown in FIG. 13, to an inflated state, as shown in FIG. 14. In the inflated state, the balloon 300 may engage walls of the bronchiole 508 to define a proximal region 1302 and a distal region 1304 within the bronchiole 508. The vent tube 1300 may permit airflow between the proximal and distal regions 1302, 1304, which may prevent the lung 500 (FIG. 5) from collapsing.

Embodiments disclosed herein include:

A. A surgical system comprising a probe and a balloon operatively coupled to the probe. The probe comprises a shaft, an antenna extending from the shaft, and a conductor extending through the shaft and the antenna and operable to deliver energy to tissue of a patient. The balloon is transitionable between an inflated state and a deflated state. In the inflated state, the balloon maintains a position of the antenna relative to the tissue and enhances energy delivery from the conductor to the tissue.

B. A surgical system comprising a probe and a balloon transitionable between an inflated state and a deflated state. The probe comprises a shaft, an antenna extending from the shaft, and a conductor extending through the shaft and the antenna and operable to deliver energy to tissue of a patient. The balloon controls a direction of energy delivered from the conductor in the inflated state.

C. A method comprising advancing a probe toward a location of tissue in a patient, the probe including a shaft, an antenna extending from the shaft, a conductor extending through the antenna, and a balloon operatively coupled to one of the shaft and the antenna. The method further comprises transitioning the balloon from a deflated state to an inflated state and thereby maintaining a position of the antenna relative to the tissue, delivering energy to the tissue with the conductor and thereby ablating a portion of the tissue, and enhancing energy delivery from the conductor to the tissue with the balloon.

Each of embodiments A, B, and C may have one or more of the following additional elements in any combination: Element 1: wherein the balloon is operatively coupled to the antenna. Element 2: wherein the balloon is operatively coupled to the shaft. Element 3: further comprising a fluid input line extending through the shaft and fluidically coupling the balloon with a fluid source, wherein the balloon is transitioned toward the inflated state by conveying a fluid to the balloon from the fluid source via the fluid input line. Element 4: further comprising a fluid output line extending through the shaft and fluidically coupled to the balloon, wherein the balloon is transitioned toward the deflated state based on fluid flowing through the fluid output line from the balloon. Element 5: further comprising a control system communicably couplable to the fluid source to control a flow rate of fluid from the fluid source through the balloon, via the fluid input and output lines, and based on the conductor delivering energy to the tissue. Element 6: wherein the balloon centers the antenna within a bronchiole in the inflated state. Element 7: wherein the balloon forces the antenna into contact within an anatomic structure in the inflated state. Element 8: wherein the balloon includes a metallic element. Element 9: wherein the metallic element is to deliver energy to the tissue. Element 10: wherein the metallic element is to block energy emitted from the conductor of the energy delivery. Element 11: wherein the balloon is operatively coupled to the antenna and extends from the antenna in a first direction, and wherein the balloon pushes the antenna in a second direction opposite the first direction as the balloon is transitioned to the inflated state. Element 12: wherein the balloon includes a metallic element operable to control the direction of energy delivered from the conductor. Element 13: wherein the metallic element is to deliver energy to the tissue. Element 14: wherein the metallic element is to block energy delivery from the conductor of the probe. Element 15: wherein the probe further includes a fluid input line extending through the shaft and fluidically coupling the balloon with a fluid source and a fluid output line extending through the shaft and fluidically coupled to the balloon, and the method further comprises controlling a flow rate of fluid from the fluid source through the balloon, via the fluid input and output lines, based on the conductor delivering energy to the tissue Element 16: wherein the balloon includes a metallic element, and enhancing energy delivery from the conductor to the tissue with the balloon comprises drawing energy emitted from the conductor in the direction of the metallic element. Element 17: wherein enhancing energy delivery from the conductor to the tissue with the balloon comprising delivering energy to the tissue with the metallic element.

By way of non-limiting example, exemplary combinations applicable to A, B, and C include: Element 1 with Element 3; Element 1 with Elements 3 and 4; Element 1 with Elements 3, 4, and 5, Element 1 with Element 6; Element 1 with Element 6; Element 1 with Element 7; Element 1 with Element 8; Element 1 with Element 9; Element 1 with Elements 8 and 9; Element 1 with Elements 8, 9, and 10; Element 2 with Element 3; Element 2 with Elements 3 and 4; Element 2 with Elements 3, 4, and 5; Element 2 with Element 6; Element 2 with Element 6; Element 2 with Element 7; Element 2 with Element 8; Element 2 with Element 9; Element 2 with Elements 8 and 9; Element 2 with Elements 8, 9, and 10; Element 3 with Element 4; Element 3 with Elements 4 and 5; Element 11 with Element 12; Element 11 with Elements 12 and 13; Element 11 with Elements 12, 13, and 14; Element 12 with Element 13; Element 12 with Element 14; Element 12 with Elements 13 and 14; Element 15 with Element 16; Element 15 with Element 16 and 17; and Element 16 with Element 17.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A surgical system, comprising:
a probe, comprising:
a shaft;
an antenna extending from the shaft; and
a conductor extending through the shaft and the antenna and operable to deliver energy to tissue of a patient; and
a balloon operatively coupled to the probe and transitionable between an inflated state and a deflated state,
wherein, in the inflated state, the balloon maintains a position of the antenna relative to the tissue and enhances energy delivery from the conductor to the tissue.

2. The surgical system of claim 1, wherein the balloon is operatively coupled to the antenna.

3. The surgical system of claim 1, wherein the balloon is operatively coupled to the shaft.

4. The surgical system of claim 1, further comprising a fluid input line extending through the shaft and fluidically coupling the balloon with a fluid source, wherein the balloon is transitioned toward the inflated state by conveying a fluid to the balloon from the fluid source via the fluid input line.

5. The surgical system of claim 4, further comprising a fluid output line extending through the shaft and fluidically coupled to the balloon, wherein the balloon is transitioned toward the deflated state based on fluid flowing through the fluid output line from the balloon.

6. The surgical system of claim 5, further comprising a control system communicably couplable to the fluid source to control a flow rate of fluid from the fluid source through the balloon, via the fluid input and output lines, and based on the conductor delivering energy to the tissue.

7. The surgical system of claim 1, wherein the balloon centers the antenna within a bronchiole in the inflated state.

8. The surgical system of claim 1, wherein the balloon forces the antenna into contact within an anatomic structure in the inflated state.

9. The surgical system of claim 1, wherein the balloon includes a metallic element.

10. The surgical system of claim 9, wherein the metallic element is to deliver energy to the tissue.

11. The surgical system of claim 9, wherein the metallic element is to block energy emitted from the conductor of the energy delivery.

12. A surgical system, comprising:
a probe, comprising:
a shaft;
an antenna extending from the shaft; and
a conductor extending through the shaft and the antenna and operable to deliver energy to tissue of a patient; and
a balloon transitionable between an inflated state and a deflated state, wherein the balloon controls a direction of energy delivered from the conductor in the inflated state.

13. The surgical system of claim 12, wherein the balloon is operatively coupled to the antenna and extends from the antenna in a first direction, and wherein the balloon pushes the antenna in a second direction opposite the first direction as the balloon is transitioned to the inflated state.

14. The surgical system of claim 12, wherein the balloon includes a metallic element operable to control the direction of energy delivered from the conductor.

15. The surgical system of claim 14, wherein the metallic element is to deliver energy to the tissue.

16. The surgical system of claim 14, wherein the metallic element is to block energy delivery from the conductor of the probe.

17. A method, comprising:
advancing a probe toward a location of tissue in a patient, the probe including:
a shaft;
an antenna extending from the shaft;
a conductor extending through the antenna; and
a balloon operatively coupled to one of the shaft and the antenna;
transitioning the balloon from a deflated state to an inflated state and thereby maintaining a position of the antenna relative to the tissue;
delivering energy to the tissue with the conductor and thereby ablating a portion of the tissue; and
enhancing energy delivery from the conductor to the tissue with the balloon.

18. The method of claim 17, wherein the probe further includes:
a fluid input line extending through the shaft and fluidically coupling the balloon with a fluid source; and
a fluid output line extending through the shaft and fluidically coupled to the balloon; and
the method further comprises controlling a flow rate of fluid from the fluid source through the balloon, via the fluid input and output lines, based on the conductor delivering energy to the tissue.

19. The method of claim 17, wherein the balloon includes a metallic element, and enhancing energy delivery from the conductor to the tissue with the balloon comprises drawing energy emitted from the conductor in the direction of the metallic element.

20. The method of claim 19, wherein enhancing energy delivery from the conductor to the tissue with the balloon comprising delivering energy to the tissue with the metallic element.
